(19) United States
(12) Patent Application Publication
(10) Pub. No.: US 2021/0128870 A1
(43) Pub. Date: May 6, 2021

(54) FELINE FOLEY CATHETER

(71) Applicant: Companion Design L.L.C., Menlo Park, CA (US)

(72) Inventor: Elliott Brenner, Menlo Park, CA (US)

(21) Appl. No.: 17/083,575

(22) Filed: Oct. 29, 2020

Related U.S. Application Data
(60) Provisional application No. 62/928,503, filed on Oct. 31, 2019.

(51) Int. Cl. A61M 25/00; A61M 25/10

(52) U.S. Cl. CPC A61M 25/0017; A61M 25/0026; A61M 2025/0063; A61M 2210/1085; A61M 2210/1089; A61M 25/10181

(57) ABSTRACT

A device, which may be a veterinary Foley-type urinary catheter for use with feline patients, and more specifically, male feline patients is described herein. The device is of sufficient size to pass through a male feline's urethra and without the need to be sutured in place to secure to the patient. The catheter may include a proximal portion configured to engage accessories, tools and instruments that may pass through the proximal portion into the catheter portion of the device. The proximal portion may include two separate tubular arms that may engage these accessories to allow fluid passage easily through the device as well as the tools and instruments. The catheter portion may be approximately 3 to 5 Fr in diameter and may further include a balloon residing at the distal portion of the catheter. The entire length of the catheter portion may be approximately 22 cm to 36 cm and include a taper toward a distal tip.

### CROSS-REFERENCE TO RELATED APPLICATIONS

A claim for priority to the Oct. 31, 2019 filing date of U.S. Provisional Patent Application No. 62/928,503, titled FOLEY CATHETER (“the '503 Provisional Application”), is hereby made pursuant to 35 U.S.C. § 119(e). The entire disclosures of the '503 Provisional Application is are hereby incorporated herein.

### FIELD

This disclosure relates generally to a veterinary catheter, more specifically, a Foley-type indwelling urinary catheter. The catheter may be a multi-lumen catheter primarily used for domesticated feline veterinary patients and even more specifically for male felines.

### RELATED ART

Many different types of urinary catheters are currently used in canine and feline veterinary patients. For domesticated male feline veterinary patients, temporary and indwelling urinary catheters vary in their particular designs however indwelling urinary catheters placed retrograde via the urethra into the bladder share the common characteristic of being secured to the patient via preputial sutures. Placement of preputial sutures has the potential for iatrogenic trauma and secondary inflammation. Current designs of indwelling urinary catheters for domesticated male feline patients do not have a self-retaining (i.e. Foley-type) inflatable balloon which may be due to the intersection of anatomical and manufacturing constraints.

Current urinary catheter designs may use a guide wire or an indwelling stylet within the catheter lumen to help to re-establish patency of the urethra as a component of treatment of urethral obstruction and associated diseases affecting the lower urinary tract in domesticated male feline veterinary patients. Previously defined diseases associated with urethral obstruction in domesticated male cats include feline interstitial cystitis, urethral plug formation, urolithiasis, urinary tract infection, neoplasia, anatomical defects, and foreign objects. The diseases may occur simultaneously and be interrelated. The current disclosure is of a feline Foley catheter composed of design aspects intended to provide a urinary catheter with functional and technical advantages in the treatment of urethral obstruction that does not need to be secured by preputial sutures and is easier to place, maintain, and remove. The current system may be applicable to the management of additional categories of diseases of male or female veterinary patients wherein patients are either too injured or ill to urinate appropriately or when closely monitoring the quantity and qualities of their urine output is important.

### SUMMARY

The following disclosure relates to a catheter system which may comprise a proximal portion with a first arm and a second arm with a catheter portion, or intermediate portion with an Fr diameter between 3 to 5, and more specification between 3 to 3.5. Additionally, a distal portion may be distal the catheter portion with a balloon portion, and a distal end, wherein the distal end may comprise a distal tip opening and a transverse opening.

The balloon portion may have a Fr diameter between 3.5 to 5.5 and the distal end may have a Fr diameter between 3 to 5. In some examples the balloon portion may have a Fr diameter between 4 to 5 and the distal end may have a Fr diameter between 3 to 3.5. The first arm may include a first opening configured to engage at least one of a number of tools including a stylet, a Luer lock, a syringe, or a barbed adaptor. The second arm may include a first opening configured to engage at least one of a stylet, a Luer lock, a syringe, a barbed adaptor, or a urine collection system.

The proximal portion may further include a molded wedge positioned between the first and second arms and include a slit passing through the body of the wedge transversely. A hook and loop closure strap may pass through the slit and may be secured to the patient and the device to hold the device in place. In other configurations, the proximal portion may include a bifurcation and a first port for urine drainage and a second port for catheter balloon inflation.

The catheter system may be between 22 cm to 36 cm in length wherein the length of the catheter portion is 20 cm to 29 cm and the length of the proximal portion is between 2 cm to 7 cm.

### BRIEF DESCRIPTION OF THE DRAWINGS

These and other features, aspects, and advantages of the present disclosure are better understood when the following disclosure is read with reference to the accompanying drawings. The following drawings illustrate what are currently considered to be specific representative configurations for carrying out the invention and are not limiting as to embodiments which may be made in accordance with the present invention. The components in the drawings are not necessarily to scale relative to each other. Like reference numerals designate corresponding parts throughout the several views.

FIG. 1 is a perspective view of one configuration of a catheter system 10 with a proximal end and a distal end with an open ended tip.

FIG. 2 is a side plan view of a configuration of a catheter system 10.

FIG. 3 is a cross-sectional view of the catheter system of FIG. 2.

FIG. 4 is a perspective view taken from the proximal end of the catheter system as disclosed herein.

FIG. 5 is a perspective view of the stylet of the catheter system of FIG. 4.

FIG. 6 is a perspective view of an exemplary hub of a catheter system.

FIG. 7 is a cross-sectional view of the hub of FIG. 6.

FIG. 8 is a perspective view of the catheter tube of the catheter system.

FIG. 9 is a cross-sectional view of FIG. 8.

FIG. 10 is a detailed cross-section view of the distal end of the catheter system as disclosed herein.

### DETAILED DESCRIPTION

The following provides a detailed description of particular embodiments of the present invention. Reference will now be made to the drawings in which the various elements of the illustrated configurations will be given numerical designations and in which the invention will be discussed so as to enable one skilled in the art to make and use the invention. It is to be understood that the following description is only exemplary of the principles of the present invention, and should not be viewed as narrowing the scope of the claims which follow, which claims define the full scope of the invention.

It will be appreciated that various aspects discussed in one drawing may be present and/or used in conjunction with the embodiment shown in another drawing, and each element shown in multiple drawings may be discussed only once. For example, in some cases, detailed description of well-known items or repeated description of substantially the same configurations may be omitted. The reason is to facilitate the understanding of those skilled in the art by avoiding the following description from being unnecessarily redundant. The accompanying drawings and the following description are provided in order for those skilled in the art to fully understand the present disclosure, and these are not intended to limit the gist disclosed in the scope of claims.

The description merely illustrates the principles of the present subject matter. It will thus be appreciated that those skilled in the art will be able to devise various arrangements that, although not explicitly described herein, embody the principles of the present subject matter and are included within its spirit and scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

Reference in the specification to “one configuration” “one embodiment,” “a configuration” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” in various places may not necessarily limit the inclusion of a particular element of the invention to a single configuration, rather the element may be included in other or all configurations discussed herein.

It should also be noted that, as used in this specification and the appended claims, singular forms such as “a,” “an,” and “the” may include the plural unless the context clearly dictates otherwise. Thus, for example, reference to “an arm” may include one or more of such arms, and reference to “the tube” may include reference to one or more of such tubes. As used herein, the term “about” is used to provide flexibility to a numerical range endpoint by providing that a given value may be “a little above” or “a little below” the endpoint while still accomplishing the function associated with the range. “Proximal” as used herein refers to an end or portion of the device that is closer to a veterinarian user, while “distal” refers to an end or portion of the device that is to be placed entirely or partially in the veterinary patient's bladder.

Sizes, proportions, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described. It is typical in the art of catheters to refer to their dimensions such as diameter in the units of “French.” For reference, 1 “French” or 1 “Fr” is equivalent to 0.33 mm.

The following description sets forth a system or catheter for treating ailments and disease in veterinary patients. The system may include a proximal end that may be configured to secure separate attachments to a catheter, or tube, that extends from the proximal end to a distal end with an open ended distal tip. As will be described in greater detail herein the use and dimensions of the system may allow for improved treatment of the veterinary patient.

The following description sets forth separate embodiments and methods for utilizing the system as well as different configurations. The design and configurations of the system provide functional and technical advantages for improved treatment of veterinary ailments and particularly feline ailments including but not limited to urethral obstruction.

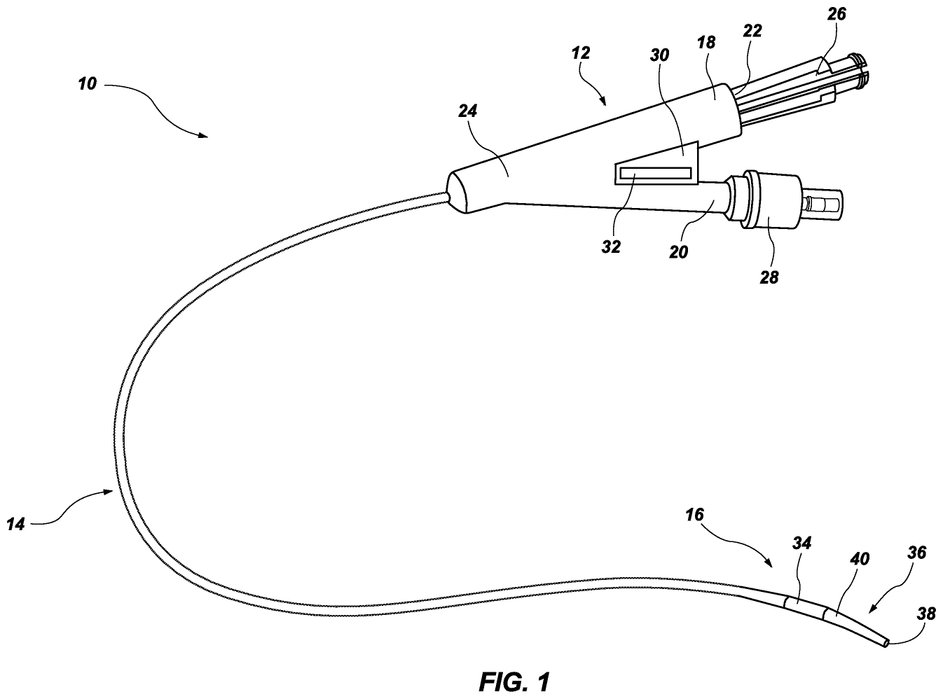

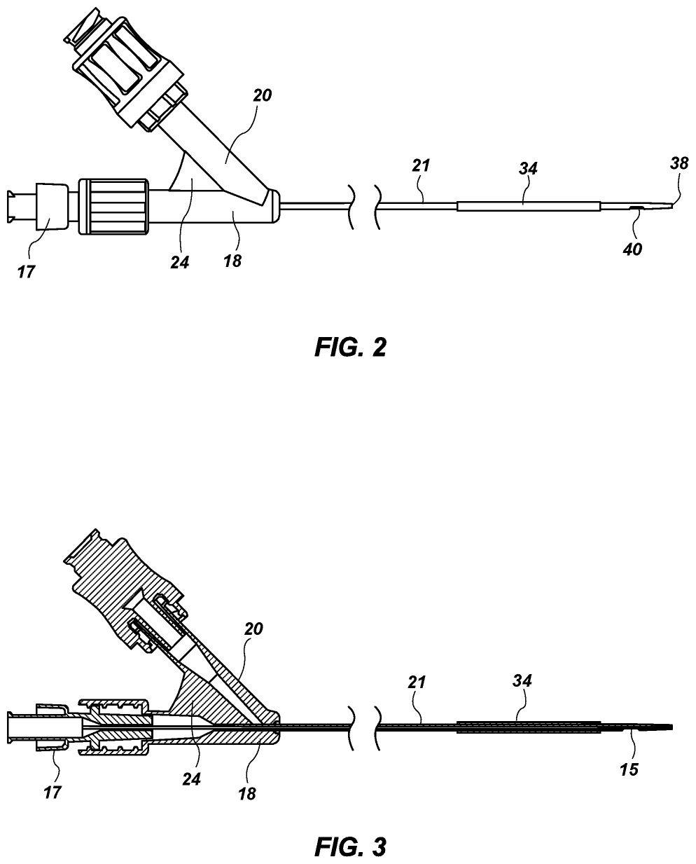

FIGS. 1-3 illustrate a catheter system 10 with a proximal portion 12, an intermediate portion or catheter tube portion 14, and a distal portion 16. The proximal portion 12 may include a junction or hub 24 with a first arm 18 and a second arm 20. The proximal portion 12 may be between about 2 cm to about 7 cm in length, and from about 2 cm to about 7 cm in height. More specifically, in some configurations, the proximal portion may be between about 3 cm to about 5 cm in length. The first arm 18 and second arm 20 may each be configured to connect to separate accessories. Each arm may include openings to allow for passage of tools or fluid and so may be essentially tubular. The first arm 18 may be in fluid communication with the open distal end of the catheter, while the second arm 20 may be in fluid communication with a balloon inflation port.

In some configurations, the catheter system 10 may include a stylet 15. The stylet may be configured to engage the first arm 18 and pass through the proximal portion 12 and through the catheter tube portion 14, of the catheter system 10. The stylet 15 may also pass through the distal portion 16. In other configurations, a stylet need not be provided. The second arm 20 may be in fluid communication with a balloon inflation port for inflating an interiorly or exteriorly positioned balloon. The first arm 18 or second arm 20, or both, may be configured to engage or be constructed with a Luer adaptor 22, or any other type of adapter suitable for the application.

One or more of the first arm 18 and/or the second arm 20 may be configured to allow for passage of a stylet or other tool and one or more of the first arm 18 and/or second arm 20 may allow for fluid flow through the catheter tube portion 14. The first arm 18 and second arm 20 may be non-parallel extensions from a hub or junction 24 of the proximal portion 12 to allow for easy access to each arm without interference with the other arm. The hub 24 of the proximal portion 12 may split into the first arm 18 and second arm 20. For example, the hub 24 may have a generally Y-shape. In other configurations, as discussed below, the first arm 18 and second arm 20 may have another junction formed to join them to the intermediate portion or primary tube 14 of the catheter tubing 21. In some configurations, the first arm 18 may be in fluid communication with a balloon inflation lumen of the primary tube 14, with the second arm 20 in fluid communication with a urine drainage lumen of the primary tube 14.

The first arm 18 of the hub 24 may be configured to engage a barbed adaptor 26 or mating attachment 26. The adaptor 26 may include a female Luer connector separate from the catheter itself and the first arm 18 may be configured to allow for engagement of this adaptor 26. A semi-rigid flushing stylet 15 with a connector 17 (such as a female Luer lock, male spin Luer lock, etc.) may be utilized with the first arm 18 as well in place of the adaptor 26. The mating attachment 26 may be attached to the first arm 18, or it may be formed integrally to the first arm 18.

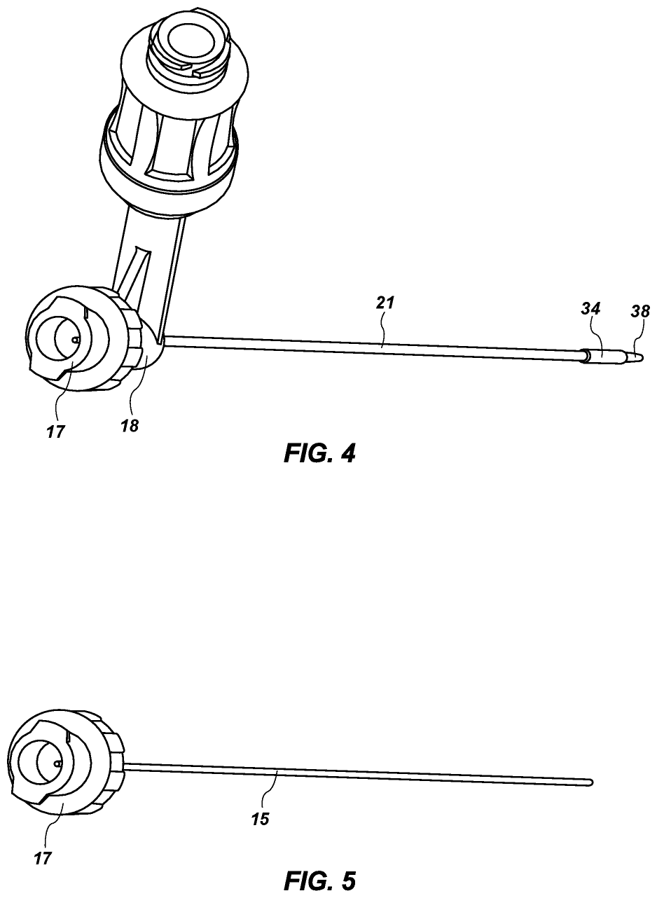

As seen in FIGS. 4-5, the flushing stylet 15 may be hollow or substantially hollow for allowing the passage of fluids therethrough. The connector 17 may be formed integrally to the stylet 15, or the stylet 15 and the connector 17 may be otherwise attached. The connector 17 may be any suitable connector to allow the stylet 15 to be removably attached to the first arm 18. The stylet 15 may be approximately 22 to 36 cm in length and may be of the largest diameter allowed by the drainage lumen 25 of catheter tubing 21. The stylet length may be chosen as a function of length of the catheter tubing. For example, the stylet may have a length that terminates about 0.5 mm to about 4 mm prior to the distal end. In one exemplary configuration, the stylet 15 has a length that terminates about 1 mm proximal from the distal tip of the catheter tubing.

In some configurations, the outside diameter of the flushing stylet 15 may be about 0.5 to about 1.0 mm, and in one specific configuration the outside diameter of the stylet 15 may be about 0.55 mm. The gauge of the stylet may be from about 18 g to about 26 g, and more specifically from 22 g to 25 g, and in some configurations may be about 24 g.

The stylet may be formed of any suitable material, and in some configurations the stylet may be formed of semi-rigid or rigid material, such as stainless steel, other metals, plastic polymer, etc. The flushing stylet 15 may also have a coating on the exterior. For example, a lubricating coating or other non-stick type coating may be used to facilitate movement of the stylet 15 in the first arm 18 and within the catheter tubing portion 14. A rigid or semi-rigid material may provide a stiffness to the catheter which may be helpful, particularly on entry or initial positioning of the catheter within the animal. A hollow stylet may allow fluids such as saline to be passed into the stylet and provide the functionality of flushing the catheter on entry. This may be useful, for example, for retrograde clearing of the urethra.

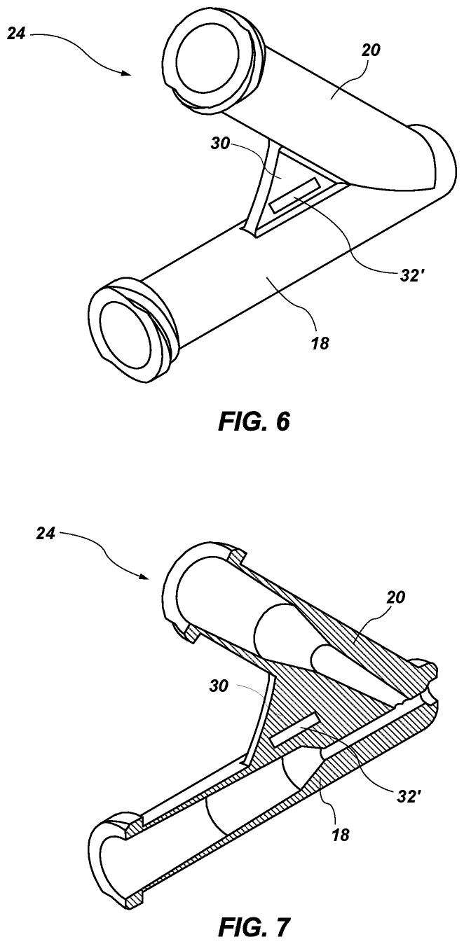

Turning now to FIGS. 6-7, the hub 24 may include the first arm 18 and a second arm 20. Each arm may be substantially hollow or have a hollow portion therethrough to allow for passage of air, fluids, instruments, etc. The hub 24 may be formed of any suitable material, such as silicone, plastic-silicone co-polymers, and other plastics. In one configuration, the hub 24 is formed of thermoplastic polymer. The first arm 18 and second arm 20 may have a length from about 20 mm to about 60 mm, and may also have a height from about 20 mm to about 60 mm. By way of example and not limitation, in one configuration, the first arm 18 has a length of 42 mm and a height of 27 mm, with the second arm 20 has a length of 46 mm and a height of 27 mm.

The second arm 20 may include a balloon inflation port with male and/or female Luer lock connectors, or other connectors, may be provided in communication with the second arm 20. The second arm 20 may allow inflation of the balloon to keep the catheter in place as described below. In some configurations, the second arm 20 can connect to a water/fluid pump to flow water or other fluid into the balloon inflation lumen of the catheter tube. The second arm 20 may include any suitable connectors, such as a female Luer lock, barb connector, etc. A mating attachment 28 may be included at a proximal end of the second arm 20. This mating attachment 28, such as a needless access male/female Luer lock valve, needless access barbed/female Luer lock valve, etc., to facilitate inflation of a balloon through the second arm 20.

Additionally, the hub 24 may include a securing mechanism 30. As seen in FIG. 1, and FIGS. 6-7, a securing mechanism 30 may be positioned at the angle where the first arm 18 and second arm 20 intersect at the hub 24. The mechanism 30 may be a simple plastic piece that is molded into the proximal portion when manufactured and may include a slit 32, or opening, that allow for passage of a hook and loop accessory with an attachment strap to secure the catheter system 10 in place without suturing to the patient. As seen in the configuration shown in FIGS. 6-7, the hub 24 may be provided with a formed portion that includes slit 32′ for receiving a hook and loop accessory with an attachment strap or other accessory. In some configurations, a strap may be provided to connect the hub 24 of the catheter to a feline patient's tail.

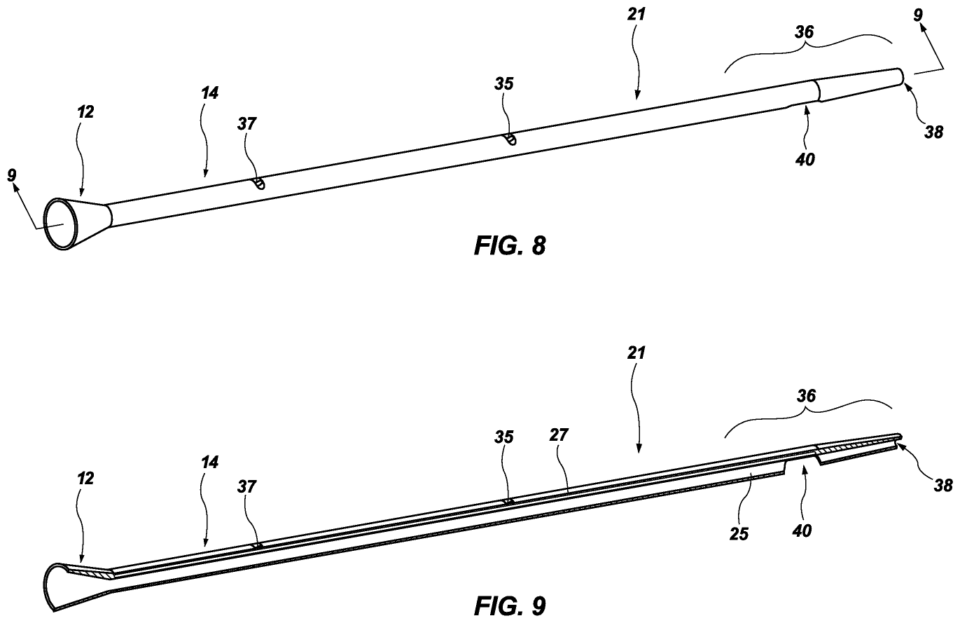

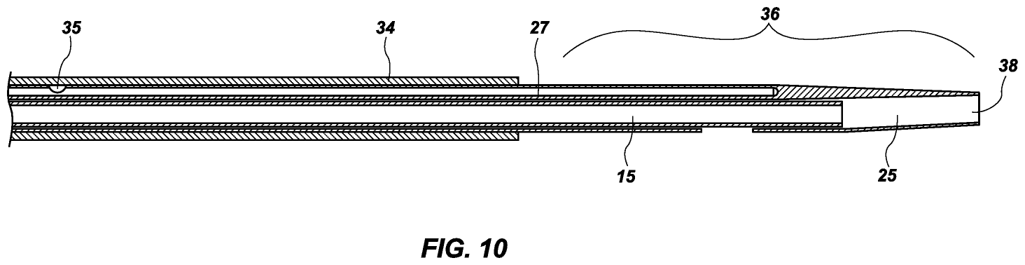

Turning now to FIGS. 8-10, the catheter tube 21 is shown (the hub 24 and associated structures have been removed for clarity). The catheter tube 21 may be a multi-lumen tube, and may include a proximal portion 12, catheter tube portion 14, as well as a distal drainage tube portion 36. The catheter tube 21 may have a step down, or shoulder, that transitions from the proximal portion 12 to the catheter tube portion 14. In other configurations, there is no step-down provided between the proximal portion 12 and the primary tube 14. The shoulder may improve the connection between the catheter tube 21 and the hub 24, and ensure that the catheter tube is not removed distally from the hub 24. The proximal end of the catheter tube 21 may be circular, or may have a non-circular profile, such as a bump or tapered extrusion.

The diameter of the catheter tube portion 14 may be between about 3 Fr to about 5 Fr. More specifically, the diameter of the catheter tube portion 14 may be between about 3 to about 4 Fr diameter. In one exemplary configuration, the diameter of the catheter tube portion 14 is 3 Fr.

In some configurations, the catheter tube portion 14 may include two lumens which are separated from each other such that they are not in fluid communication with each other. For example, the catheter tube portion 14 may include a urine drainage lumen 25 and a balloon inflation lumen 27. The urine drainage lumen 25 may be larger in diameter than the balloon inflation lumen 27. As discussed above, the urine drainage lumen 25 may be in fluid communication with the first arm 18, and the balloon inflation lumen 27 may be in fluid communication with the second arm 20. The urine drainage lumen 25 may also be configured to receive a stylet, such as flushing stylet 15 of FIG. 5.

The catheter tube 21 may be made from any suitable known material, such as silicone, polyurethane silicone co-polymer, polyvinyl chloride, etc. In one configuration the catheter tube is made from thermoplastic polyurethane. The length of the catheter tube shaft may vary depending on the desired use of the catheter system 10. For example, the length of the shaft of the catheter tube portion 14 proximal to the balloon 34 may be from about 15.5 cm to about 27.5 cm. In one configuration the length may be about 24 cm.

The distal portion 16 may be a continuation of the catheter tube portion 14 and may include a balloon 34 within the catheter or attached to the exterior of the catheter tube 21. The balloon 34 is deflated for insertion into a patient. The balloon inflation lumen 27 extends within the catheter tubing 21 from the proximal portion 12, through the catheter tube portion 14, and terminates before the distal tip 38. The balloon inflation lumen 27 is in fluid communication with balloon 34 and second arm 20. For example, a first slit or opening 35 from the exterior of the catheter tube 21 to the balloon inflation lumen 27 may be provided near the distal end, and a second slit or opening 37 from the exterior of the catheter tube 21 to the balloon inflation lumen 27 may be provided near the proximal end. The first slit 35 may be located where the balloon 34 covers the exterior of the catheter 21, allowing the balloon inflation lumen 27 to be in fluid communication with the balloon 34. The second slit 37 may be located where the second arm 20 covers the exterior of the catheter 21, allowing the balloon inflation lumen 27 to be in fluid communication with the second arm 20.

The balloon inflation lumen 27 may terminate at any point before the open distal tip 38. In some configurations, the balloon inflation lumen terminates before the distal drainage tube portion 36, such that the distal drainage tube portion is a single-lumen distal drainage tube portion. In other configurations, the balloon inflation lumen 27 extends partially into the distal drainage tube portion 36 before terminating before open distal tip 38.

Any suitable balloon may be attached to the exterior of the catheter tube 21. In some configurations, the balloon may have a diameter between about 3.5 Fr to 5.5 Fr. In one configuration, the balloon's diameter may be 3.5 Fr. The length of the balloon portion may be between about 0.5 cm to about 1.5 cm with a volume of about 0.25 mL to about 1 mL. In one configuration the length is about 10 mm and the volume of the balloon 34 is about 0.5 mL. The balloon may have a spherical or semi-spherical shape, or any other suitable shape. Similarly, the balloon may be made from any suitable material such as plastics and co-polymers, polyurethane, polyurethane-silicone co-polymer, silicone, etc. The balloon may be filled as desired based on the application, and may be filled, for example, with a saline solution or sterile water. In one configuration, the balloon is made of silicone and filled with a 0.9% saline solution.

A distal end or distal drainage tube portion 36 may be between 1 to 3 cm in length and may be positioned distal to the balloon 34 or directly distal to the balloon 34. The distal end portion 36 may have a diameter similar to the intermediate portion, between about 3 Fr diameter to about 5 Fr diameter. More specifically, the distal end may have a diameter between about 3 Fr to about 4 Fr. Additionally, a distal tip 38 may include a taper, such as a radial taper, a bullet nose taper, etc. The distal end portion 36 may have an open distal tip 38, such that the first drainage lumen is open to the open distal tip.

In some configurations, the distal drainage portion 36 includes one or more fenestrations or transverse openings 40 proximal the open distal tip 38. The transverse opening(s) may be in fluid communication with the first drainage lumen, and thus may facilitate passage of fluids through the first drainage lumen of the catheter system 10. For example, 1-2 transverse openings may be provided, from about 2 mm to about 8 mm proximal from the open distal end 38, each transverse opening having an opening size of about 0.5 mm to about 2 mm. The openings may have a circular shape, semi-circular shape, elliptical shape, etc. In one exemplary configuration, a single transverse opening 40 may be provided, with a semi-circular shape and a 1 mm diameter, approximately 4 mm proximal from the open distal tip 38.

The catheter tubing 21 may be comprised of many different materials which may include, but are not limited to, silicone and polyurethane, but other polymers, and metals are both considered and contemplated herein. Additionally, the catheter tubing 21 may be radiopaque and may have a coating. For example, the catheter tubing may have radiopaque banding or markings. Some configurations may have radiopaque additive only on the distal end to view placement of the distal end of the catheter tubing 21. In other configurations, the tubing 21 may be provided with exterior printed markings, such as centimeter demarcations, Fr size, etc. The catheter tubing 21 may also have a coating or jacketing as desired, such as an antimicrobial coating, lubricating coating, etc.

Ultimately the present disclosure is intended to be of sufficient diameter but also narrow enough diameter for domesticated feline patients that is sufficiently narrow to pass through a male domesticated feline urethra. The system described herein is also intended to engage a plurality of accessories for use with the catheter system 10. The accessories include, but are not limited to a stylet, Luer locks, barbed adaptors, accessory placement hook and loop closure strap, urine collection system, etc. The method of manufacture of how to make a catheter as disclosed herein is fully considered and contemplated.

In use in a veterinary setting for a male feline veterinary patient for the treatment of urethral obstruction and associated diseases affecting the lower urinary tract, the clinician would first anesthetize the patient, as deemed appropriate based upon the patient's circulatory and metabolic status, and perform sterile preparation of the preputial region in advance of catheter insertion. The clinician may then select the appropriately sized catheter system inclusive of an integral flushing stylet component based on diameter and length. Next, the clinician may prepare the catheter for entry into the urethra by first inserting a stylet 15 into the first arm. The flushing stylet 15 includes a connector 17 to be attached to the mating attachment 26 of the first arm 18. In other configurations, a stylet 15 may be placed into the drainage lumen 25 during manufacturing and/or the catheter system 10 may be sold with a flushing stylet 15 already in place within the first arm 18 and drainage lumen 25. That is, the catheter system 10 may be sold with a flushing stylet 15 already inserted inside the first arm within sterile packaging.

After preparing the catheter as needed, the clinician may then insert the distal tip into the distal end of the urethra. As the clinician moves the distal tip proximally towards the patient's bladder, the clinician may also pass fluid into the flushing stylet 15 as desired. The flushing stylet 15 may provide some rigidity to the catheter system 10 to help insertion. Any types of obstructions, etc., may be retrogradely removed by the fluids being passed through the flushing stylet.

When the clinician has the distal drainage tube portion 36 and balloon 34 within the bladder of the patient, fluid can then be used to fill the balloon 34 via the second balloon inflation lumen 27. The inflated balloon may keep the distal drainage tube portion 36 of the catheter system 10 in place within the patient's bladder without the need for preputial sutures. Following balloon inflation, the stylet 15 will be removed. Drainage of the bladder may occur through the open distal tip and/or one or more transverse openings 40 in the distal portion 36 of the catheter tubing 21. If desired, the hub 24 may be attached to the patient, such as through a strap or other attachment means passed through the slit 32 of the hub.

Various aspects are disclosed herein. Aspect A: a method for implanting a multi-lumen catheter into a bladder of a feline veterinary patient without the need for sutures, the multi-lumen catheter comprising a one-piece multi-lumen tube portion having a plurality of integrally formed lumens with a diameter of about 3 Fr to about 5 Fr, a proximal end portion including a first arm in fluid connection with a first of the plurality of integrally formed lumens and a second arm in fluid connection with a second of the plurality of integrally formed lumens, the first arm having a hollow flushing stylet located therein, and a distal end portion including a single-lumen portion with an open distal end, the second of the plurality of integrally formed lumens terminating before the open distal end, and the second of the plurality of integrally formed lumens in fluid communication with a balloon, the method comprising: inserting the open distal end of the multi-lumen catheter into the distal end of a patent's urethra; guiding the distal end portion of the multi-lumen catheter and the balloon such that they are fully within the bladder; and after the distal end portion of the multi-lumen catheter and the balloon are in the bladder, filling the balloon by forcing fluid into the second arm of the proximal end portion.

Aspect B: The method of aspect A, wherein the proximal end portion further comprises a hub connecting the first arm and second arm, the hub further comprising a cut-out for receiving a connection means to connect the hub to a tail of the feline veterinary patient, and wherein the method further comprises the step of attaching the hub to the tail of the patient.

Aspect C: The method of aspect A or B, wherein the method further comprises forcing fluid into the hollow flushing stylet of the first arm while guiding the catheter proximally through the urethra to the bladder to retrogradely remove at least one obstruction.

Aspect D: The method of Aspects A, B or C, wherein the distal end portion further comprises a transverse opening in fluid communication with the first of the plurality of integrally formed lumens, and wherein the method further comprises the step of draining fluid from the bladder through at least one of the open distal end and the transverse opening.

Aspect E: A method of retrogradely removing an obstruction from a urethra of a feline, the method comprising: selecting a multi-lumen catheter comprising a one-piece multi-lumen tube portion having a plurality of integrally formed lumens with a diameter of about 3 Fr to about 5 Fr, a proximal end portion including a first arm in fluid connection with a first of the plurality of integrally formed lumens and a second arm in fluid connection with a second of the plurality of integrally formed lumens, the first arm having a hollow flushing stylet located therein, and a distal end portion including a single-lumen with an open distal end; inserting the distal end portion of the multi-lumen catheter through a distal end of a patent's urethra; and forcing fluid into the hollow flushing stylet of the first arm while guiding the multi-lumen catheter proximally through the urethra to retrogradely remove at least one obstruction.

Aspect F: The method of Aspect E, further comprising the steps of: guiding the distal end portion of the multi-lumen catheter and the balloon such that they are fully within the bladder; and after the distal end portion of the multi-lumen catheter and the balloon are in the bladder, filling the balloon by forcing fluid into the second arm of the proximal end portion.

Aspect G: The method of Aspects E or F, wherein the distal end portion further comprises a transverse opening in fluid communication with the first of the plurality of integrally formed lumens, and wherein the method further comprises the step of draining fluid from the bladder through at least one of the open distal end and the transverse opening.

Aspect H: The method of Aspects E, F, or G, wherein the proximal end portion further comprises a hub connecting the first arm and second arm, the hub further comprising a cut-out for receiving a connection means to connect the hub to a tail of the feline veterinary patient, and wherein the method further comprises the step of attaching the hub to the tail of the patient.

Aspect I: A multi-lumen catheter system comprising: a proximal portion including a first arm and a second arm; a one-piece multi-lumen catheter tube portion including a first integrated lumen and a second integrated lumen, the first arm in fluid connection with the first integrated lumen and the second arm in fluid connection with the second integrated lumen, the one-piece multi-lumen catheter tub portion having a diameter between about 3 Fr to about 5 Fr; wherein the one-piece multi-lumen catheter tube portion comprises an open distal tip, the second integrated lumen terminating proximal to the open distal tip and the first integrated lumen in fluid communication with the open distal tip; wherein the one-piece multi-lumen catheter tube portion comprises a distal end portion including a balloon portion and a distal drainage tube portion, the balloon portion including: the first integrated lumen and second integrated lumen, wherein the second integrated lumen is in fluid communication with a balloon and wherein the second integrated lumen terminates before the open distal tip, the balloon to enable placement of the distal end portion in a feline veterinary patient's bladder without the need for sutures; and the distal drainage tube portion including the open distal tip and a transverse opening in fluid communication with the first integrated lumen.

Aspect J: The multi-lumen catheter system of Aspect I, wherein the multi-lumen catheter system further comprises a hollow flushing stylet removably connected to the first arm.

Aspect K: The multi-lumen catheter system of Aspect I or Aspect J, wherein the distal drainage tube portion is tapered toward the open distal tip.

Aspect L: The multi-lumen catheter system of any of Aspect I, J, or K wherein the balloon portion has a FR diameter between 3.5 to 5.5.

Aspect M: The multi-lumen catheter system of any of Aspect I, J, K, or L, wherein the distal end portion has a FR diameter between 3 to 5.

Aspect N: The multi-lumen catheter system of any of Aspect I, J, K, L, or M, wherein the first arm comprises a first opening configured to engage at least one of a stylet, a Luer lock, a syringe, and a barbed adaptor.

Aspect O: The multi-lumen catheter system of any of Aspect I, J, K, L, M, or N, wherein the second arm comprises a first opening configured to engage at least one of a stylet, a Luer lock, a syringe, a barbed adaptor, and a urinary collection system.

Aspect P: The multi-lumen catheter system of any of Aspect I, J, K, L, M, N, or O, the proximal portion further comprising a securing mechanism comprising a hub portion connecting the first and second arms, and wherein the hub portion further comprises a slit, and a hook and loop closure strap configure to pass through the slit.

Aspect Q: The multi-lumen catheter system of any of Aspect I, J, K, L, M, N, O, or P, wherein a length of the a one-piece multi-lumen catheter tube portion is about 20 cm to 29 cm, and the length of the proximal portion is about 2 cm to 7 cm.

Although the foregoing disclosure provides many specifics, these should not be construed as limiting the scope any of the ensuing claims. For example, although the disclosure was described with respect to feline applications, it may have applications for other animals as well. Other embodiments may be devised which do not depart from the scopes of the claims. Features from different embodiments may be employed separately or in combination. Accordingly, all additions, deletions and modifications to the disclosed subject matter that fall within the scopes of the claims are to be embraced thereby. The scope of each claim is indicated and limited only by its plain language and the full scope of available legal equivalents to its elements.

What is claimed:

1. A method for implanting a multi-lumen catheter into a bladder of a feline veterinary patient without the need for sutures, the multi-lumen catheter comprising a one-piece multi-lumen tube portion having a plurality of integrally formed lumens with a diameter of about 3 Fr to about 5 Fr, a proximal end portion including a first arm in fluid connection with a first of the plurality of integrally formed lumens and a second arm in fluid connection with a second of the plurality of integrally formed lumens, the first arm having a hollow flushing stylet located therein, and a distal end portion including a single-lumen portion with an open distal end, the second of the plurality of integrally formed lumens terminating before the open distal end, and the second of the plurality of integrally formed lumens in fluid communication with a balloon, the method comprising:
inserting the open distal end of the multi-lumen catheter through a distal end of a patent's urethra;
guiding the distal end portion of the multi-lumen catheter and the balloon such that they are fully within the bladder; and
after the distal end portion of the multi-lumen catheter and the balloon are in the bladder, filling the balloon by forcing fluid into the second arm of the proximal end portion.

2. The method of claim 1, wherein the proximal end portion further comprises a hub connecting the first arm and second arm, the hub further comprising a cut-out for receiving a connection means to connect the hub to a tail of the feline veterinary patient, and wherein the method further comprises the step of attaching the hub to the tail of the patient.

3. The method of claim 1, wherein the method further comprises forcing fluid into the hollow flushing stylet of the first arm while guiding the catheter proximally through the urethra to the bladder to retrogradely remove at least one obstruction.

4. The method of claim 1, wherein the distal end portion further comprises a transverse opening in fluid communication with the first of the plurality of integrally formed lumens, and wherein the method further comprises the step of draining fluid from the bladder through at least one of the open distal end and the transverse opening.

5. A method of retrogradely removing an obstruction from a urethra of a feline, the method comprising:
selecting a multi-lumen catheter comprising a one-piece multi-lumen tube portion having a plurality of integrally formed lumens with a diameter of about 3 Fr to about 5 Fr, a proximal end portion including a first arm in fluid connection with a first of the plurality of integrally formed lumens and a second arm in fluid connection with a second of the plurality of integrally formed lumens, the first arm having a hollow flushing stylet located therein, and a distal end portion including a single-lumen with an open distal end;
inserting the distal end portion of the multi-lumen catheter through a distal end of a patent's urethra; and
forcing fluid into the hollow flushing stylet of the first arm while guiding the multi-lumen catheter proximally through the urethra to retrogradely remove at least one obstruction.

6. The method of claim 5, further comprising the steps of:
guiding the distal end portion of the multi-lumen catheter and the balloon such that they are fully within the bladder; and
after the distal end portion of the multi-lumen catheter and the balloon are in the bladder, filling the balloon by forcing fluid into the second arm of the proximal end portion.

7. The method of claim 6, wherein the distal end portion further comprises a transverse opening in fluid communication with the first of the plurality of integrally formed lumens, and wherein the method further comprises the step of draining fluid from the bladder through at least one of the open distal end and the transverse opening.

8. The method of claim 5, wherein the proximal end portion further comprises a hub connecting the first arm and second arm, the hub further comprising a cut-out for receiving a connection means to connect the hub to a tail of the feline veterinary patient, and wherein the method further comprises the step of attaching the hub to the tail of the patient.

9. A multi-lumen catheter system comprising:
a proximal portion including a first arm and a second arm;
a one-piece multi-lumen catheter tube portion including a first integrated lumen and a second integrated lumen, the first arm in fluid connection with the first integrated lumen and the second arm in fluid connection with the second integrated lumen, the one-piece multi-lumen catheter tub portion having a diameter between about 3 Fr to about 5 Fr;
wherein the one-piece multi-lumen catheter tube portion comprises an open distal tip, the second integrated lumen terminating proximal to the open distal tip and the first integrated lumen in fluid communication with the open distal tip;
wherein the one-piece multi-lumen catheter tube portion comprises a distal end portion including a balloon portion and a distal drainage tube portion, the balloon portion including:
the first integrated lumen and second integrated lumen, wherein the second integrated lumen is in fluid communication with a balloon and wherein the second integrated lumen terminates before the open distal tip, the balloon to enable placement of the distal end portion in a feline veterinary patient's bladder without the need for sutures; and
the distal drainage tube portion including the open distal tip and a transverse opening in fluid communication with the first integrated lumen.

10. The multi-lumen catheter system of claim 9, wherein the multi-lumen catheter system further comprises a hollow flushing stylet removably connected to the first arm.

11. The multi-lumen catheter system of claim 9, wherein the distal drainage tube portion is tapered toward the open distal tip.

12. The multi-lumen catheter system of claim 9 wherein the balloon portion has a Fr diameter between 3.5 to 5.5.

13. The multi-lumen catheter system of claim 9 wherein the distal end portion has a Fr diameter between 3 to 5.

14. The multi-lumen catheter system of claim 9 wherein the first arm comprises a first opening configured to engage at least one of a stylet, a Luer lock, a syringe, and a barbed adaptor.

15. The multi-lumen catheter system of claim 9 wherein the second arm comprises a first opening configured to engage at least one of a stylet, a Luer lock, a syringe, a barbed adaptor, and a urinary collection system.

16. The multi-lumen catheter system of claim 9, the proximal portion further comprising a securing mechanism comprising a hub portion connecting the first and second arms, and wherein the hub portion further comprises a slit, and a hook and loop closure strap configure to pass through the slit.

17. The multi-lumen catheter system of claim 9, wherein a length of the one-piece multi-lumen catheter tube portion is about 20 cm to 29 cm, and the length of the proximal portion is about 2 cm to 7 cm.
